(19) United States
(12) Patent Application Publication
(10) Pub. No.: US 2015/0105756 A1
(43) Pub. Date: Apr. 16, 2015

(54) URINE COLLECTION ASSEMBLY AND METHOD

(71) Applicants: Daniel E. O'Brien, Calry (IE); Martin P. Creaven, Ballina (IE); Michael G. Murray, Ballina (IE); Bent Jensen, Haarlev (DK); David Hannon, Ballina (IE)

(72) Inventors: Daniel E. O'Brien, Calry (IE); Martin P. Creaven, Ballina (IE); Michael G. Murray, Ballina (IE); Bent Jensen, Haarlev (DK); David Hannon, Ballina (IE)

(73) Assignee: Hollister Incorporated, Libertyville, IL (US)

(21) Appl. No.: 14/007,283

(22) PCT Filed: Mar. 13, 2012
(86) PCT No.: PCT/US2012/028862

§ 371 Date: Apr. 7, 2014

Related U.S. Application Data
(60) Provisional application No. 61/467,679, filed on Mar. 25, 2011.

(51) Int. Cl. A61F 5/451; B65B 7/02; B65B 55/22

(52) U.S. Cl. CPC A61F 5/451; B65B 55/22; B65B 7/02
USPC 604/544; 29/428

(57) ABSTRACT

The urine collection assembly and method includes a catheter having a proximal insertion end and a distal end. The distal end of the catheter has only a cylindrical barrel portion of a tapered funnel integral therewith. The urine collection assembly further includes a urine collection bag which includes a cylindrical attachment port. The cylindrical barrel portion is sealed in the cylindrical attachment port and permits urine to pass into the urine collection bag.

### FIELD OF THE DISCLOSURE

The present disclosure relates generally to urine collection assemblies and methods for collecting urine and, more particularly, to a closed urine collection assembly and method of manufacturing a closed urine collection assembly utilizing a conventional catheter having a tapered funnel.

### BACKGROUND OF THE DISCLOSURE

Intermittent urinary catheterization is a good option for many users who suffer from various abnormalities of the urinary system. A commonly encountered situation is where single use, individually packaged, sterile catheters are used. An important criterion for any single use product is the cost of the product, i.e., a less expensive product is desired and valued.

It is also common for catheters to be provided with a surface treatment using a lubricant to reduce friction in order to allow for easier and less traumatic insertion of the catheter. Currently, there are two major categories of catheters for the drainage of urine from the bladder which have lubricated surfaces, i.e., gel lubricated catheters and hydrophilic coated catheters.

The gel lubricated catheters are made easier to insert by application to the catheter surface of a water-based gel that can be applied by the user or health care provider, or more conveniently, it can be supplied with the packaged catheter. Typically, a system is provided with the packaged catheter to apply the gel to the surface of the catheter. This system may be one where the gel is placed on the catheter surface before or during the catheter packaging operation or one where the gel is applied to the catheter surface as the catheter is inserted by the user or health care provider.

In a hydrophilic coated catheter, the catheter is provided with a thin hydrophilic coating which is adhered to the outer surface of the catheter. When this coating is activated by swelling in contact with a hydrating agent such as water, it provides a surface having a lubricious and extremely low coefficient of friction. In earlier versions, a sterile, individually packaged single use catheter is provided in a dry state, and the user or health care provider opens the package and pours water into the package to hydrate the coating. After doing so, the user or health care provider would wait 30 seconds and remove the catheter from the package ready for insertion.

A more recent version of the hydrophilic coated catheter is where the catheter is provided in a package that already contains enough loose liquid water to cause it to be fully immersed. For this product, the user simply opens the package and removes the catheter ready for insertion without the need to add water and wait 30 seconds. Other products provide the amount of liquid water necessary for immersion of the catheter, e.g., in a separate compartment of the package. With these products, the separate compartment is opened to allow the liquid immersion water to enter the catheter-containing chamber for direct contact with the hydrophilic coated surface. Depending on the product, and on the amount of water in the separate chamber, the user may be asked to manipulate the package to bathe the catheter surface in the hydrating liquid in order to activate the hydrophilic coating on the catheter surface before the catheter is removed from the package ready for insertion by the user or health care provider.

Still more recently, the need for liquid immersion of a catheter having a hydrophilic coating or surface has been eliminated. In commonly owned U.S. Pat. No. 7,380,658, it is taught to use a vapor atmosphere within a package containing such a catheter in order to hydrate the hydrophilic coating or surface of the catheter. In some instances, it is desirable to have an entire urine collection assembly which includes both a catheter and a urine collection bag. Such a urine collection assembly makes it possible to have an entirely closed system wherein, following insertion of the catheter through the urethra and into the bladder, urine can be drained from the bladder through the catheter and directly into the urine collection bag. Following use of the urine collection assembly, the catheter can be withdrawn and the assembly can be discarded, e.g., after draining urine from the bag.

As will be appreciated, the provision of an entire urine collection assembly necessarily increases the overall cost. Since for most users it is necessary to undergo catheterization frequently, cost is an important factor to be taken into consideration. As a result, any possible reduction of cost of a urine collection assembly makes its use more desirable to more users.

In addition to cost, there are other factors that are important in providing a useful and desirable urine collection assembly. For instance, it would be desirable for such an assembly to comprise a closed system which thereby substantially eliminates any possibility of leakage at the juncture of the catheter and the collection bag. Also, it would be desirable to utilize a hydrophilic coating or surface on the catheter due to the highly lubricious nature of such coatings or surfaces and the ability to eliminate use of messy gels. Further, it would be desirable to avoid utilization of solvents or adhesives in the manufacture of a urine collection assembly in order to ensure potentially dangerous solvent or adhesive vapors are not present in the urine collection bag. Additionally, it would be desirable for the urine collection bag to include an anti-reflux feature and for the entire urine collection assembly to be such as to permit relatively compact packaging.

### SUMMARY OF THE DISCLOSURE

Accordingly, the disclosure is directed to a urine collection assembly comprising a catheter and a urine collection bag. The catheter has a proximal insertion end and a distal end having a portion of a tapered funnel integrally associated therewith. The portion of the tapered funnel associated with the distal end of the catheter comprises only a cylindrical barrel portion.

The urine collection bag has a cylindrical attachment port and the cylindrical barrel portion is sealed in the cylindrical attachment port. In this manner, urine being drained from the bladder by the catheter can pass directly into the urine collection bag through a sealed joint between the catheter and the urine collection bag. Preferably, the urine collection assembly including the catheter and urine collection bag is provided in a packaged, ready-to-use condition.

In an exemplary embodiment, the urine collection bag is formed of a flexible sheet material and the attachment port is sealed into the periphery of the urine collection bag. The flexible sheet material can advantageously comprise two generally rectangular sheets of EVA/polyethylene film sealed substantially entirely about the peripheral edges thereof except where the attachment port is located as will be explained in greater detail below. Further, the cylindrical attachment port is preferably a co-extruded tube which is formed to have an EVA outer surface and a PVC inner surface.

With the cylindrical attachment port formed in this manner, the cylindrical barrel portion which is also conventionally formed of PVC is preferably sealed to the inner surface of the co-extruded tube by spin welding.

In an exemplary embodiment, the catheter is disposed externally of the urine collection bag and includes a hydrated hydrophilic outer surface. Advantageously, the catheter can also include an introducer at or near the proximal insertion end of the catheter and a sleeve substantially covering the catheter. Still further, the urine collection assembly preferably includes an anti-reflux valve sealed to the cylindrical attachment port within the urine collection bag.

In another respect, the disclosure is directed to a method of manufacturing a urine collection assembly. The method includes the step of providing a catheter having a proximal insertion end and a distal end having a tapered funnel including a cylindrical barrel portion and a conical portion integrally associated therewith. It also includes the step of cutting off the conical portion of the tapered funnel and leaving only the cylindrical barrel portion of the tapered funnel integrally associated with the distal end of the catheter. In accordance with the method, a urine collection bag is provided having a cylindrical attachment port defining an opening sized to receive the cylindrical barrel portion of the tapered funnel in size for size fashion. The method also includes the step of placing the cylindrical barrel portion of the tapered funnel into the opening defined by the cylindrical attachment port in size for size fashion. The method further includes the step of sealing the cylindrical barrel portion of the tapered funnel within the opening defined by the cylindrical attachment port of the urine collection bag.

Preferably, the method includes the steps of forming the urine collection bag of a flexible sheet material and sealing the attachment port into the sheet material. It may also advantageously include the step of using two sheets of EVA/polyethylene film for the flexible sheet material and sealing the two sheets about the peripheral edges thereof. It may also preferably include the step of providing a co-extruded tube having an EVA outer surface and a PVC inner surface for the cylindrical attachment port. In addition, the method may advantageously include the cylindrical barrel portion being formed of PVC and the sealing step being performed by spin welding.

The spin welding step is preferably performed by holding the catheter in a stationary position and spinning the urine collection bag relative to the catheter. The spin welding step may advantageously include spinning the urine collection bag relative to the catheter at a rotational speed of between 1900 and 3000 RPMs. Also, the spin welding step preferably includes spinning the urine collection bag relative to the catheter for between 14 and 16 revolutions.

Preferably, the method further includes the step of providing the urine collection bag in a generally rectangular shape, folding the urine collection bag at least once generally perpendicular to the direction of the catheter, performing the spin welding step, folding the urine collection bag at least once in the direction of the catheter, and banding the urine collection bag in a folded condition.

As for other aspects of the method, it can include the step of providing an introducer at or near the proximal insertion end of the catheter and a sleeve substantially covering the catheter. Additionally, the method can include the step of sealing an anti-reflux valve to the cylindrical attachment port within the urine collection bag to prevent reverse urine flow into the catheter.

In addition to the foregoing, the method can also comprise providing a packaged ready-to-use urine collection assembly which includes the step of providing a catheter having a hydrophilic outer surface to be hydrated prior to using the catheter, providing a gas impermeable package capable of receiving the catheter and the urine collection bag therein, placing the catheter, the urine collection bag and a hydrating agent into the gas impermeable package, and sealing the gas impermeable package with the catheter, the urine collection bag and the hydrating agent therein.

Other objects, advantages and features of the present disclosure will become apparent from a consideration of the following specification taken in conjunction with the accompanying drawings.

### BRIEF DESCRIPTION OF THE DRAWINGS

FIG. 1 is a top plan view of a urine collection assembly including a catheter and a urine collection bag in accordance with the present disclosure;

FIG. 2 is a front elevational view of the urine collection assembly including the catheter and the urine collection bag which is illustrated in FIG. 1;

FIG. 3 is a front elevational view of the urine collection assembly which is illustrated in FIG. 2 with the urine collection bag partially unfolded;

FIG. 3A is an enlarged detail view of a cylindrical barrel portion on a catheter within a cylindrical attachment port in the urine collection bag;

FIG. 3B is an enlarged detail view of the cylindrical portion of a tapered funnel on the catheter remaining after cutting off a conical portion;

FIG. 4 is a front elevational view of a catheter having a conventional tapered funnel which includes a cylindrical portion and a conical portion;

FIG. 5 is a top plan view of a urine collection bag for use with a catheter in the urine collection assembly which is illustrated in FIG. 1;

FIG. 5A is an enlarged detail view of the portion of the urine collection bag of FIG. 5 in which a cylindrical attachment port is located;

FIG. 6 is a perspective view illustrating a urine collection assembly after it is removed from a package and is in a ready-to-use condition;

FIGS. 7A-7E illustrate the series of steps in folding the urine collection bag before a final folding step into the configuration illustrated in FIGS. 1 and 2; and

FIGS. 8A-8G illustrate an apparatus for performing a method of manufacturing a urine collection assembly in accordance with the present disclosure.

### DETAILED DESCRIPTION OF THE DISCLOSURE

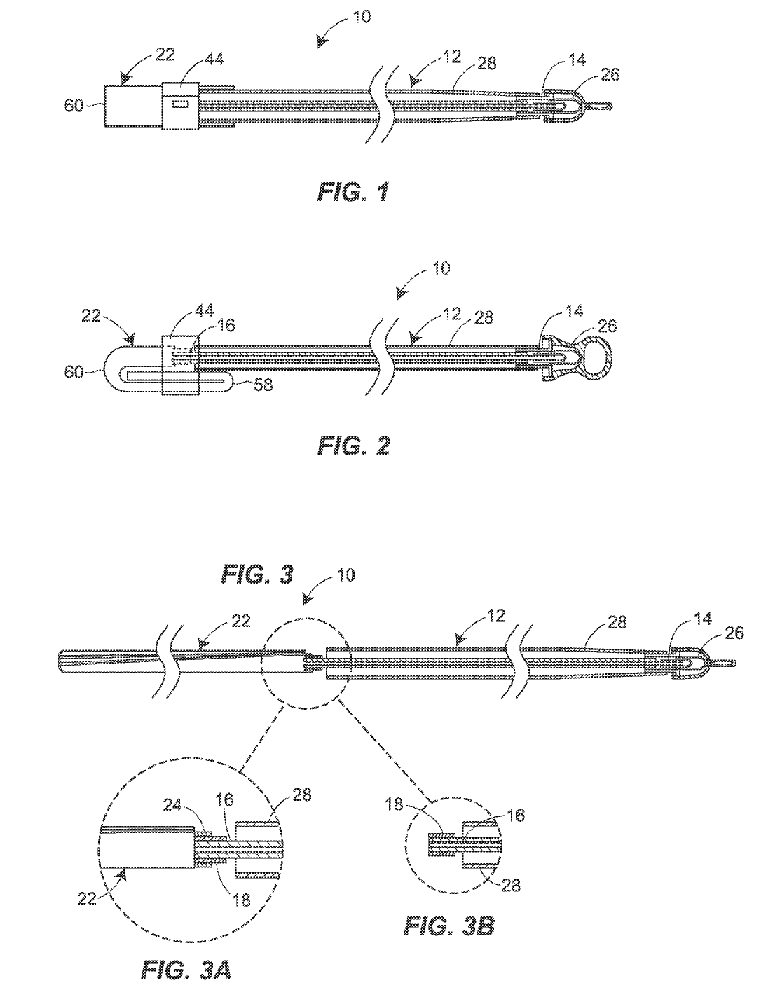

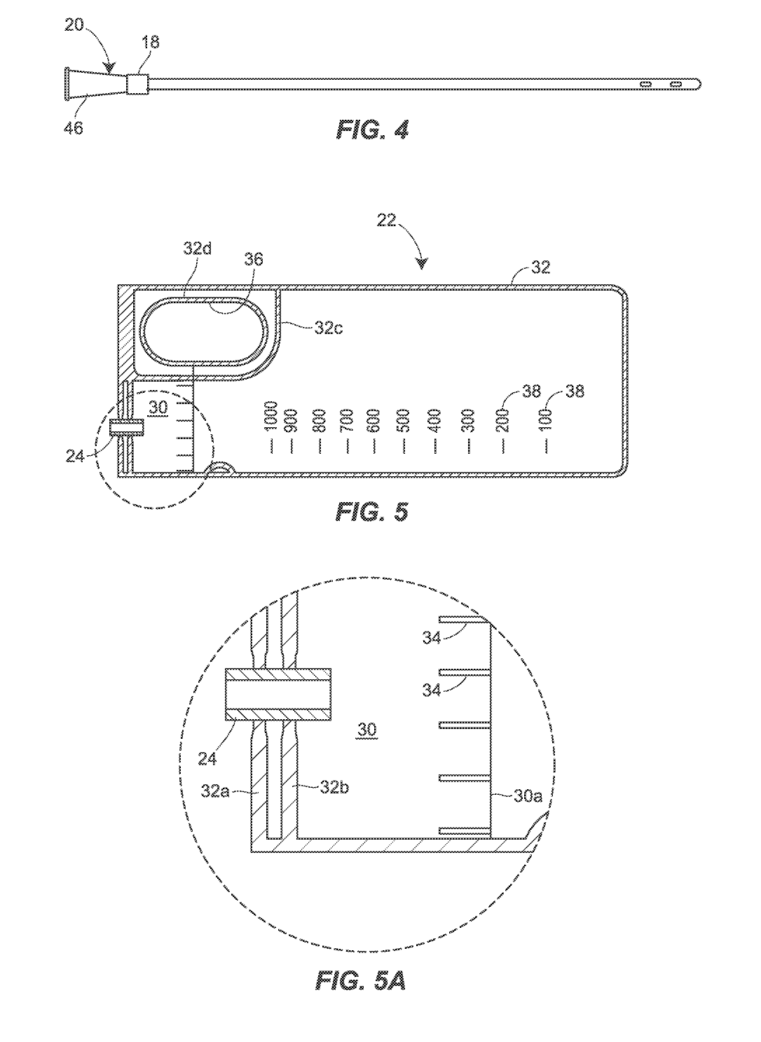

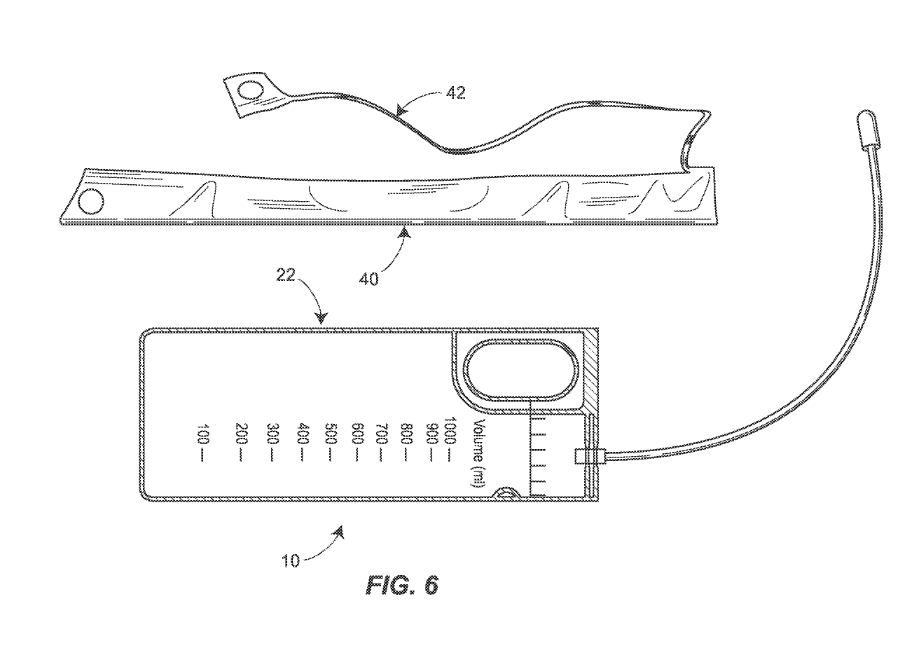

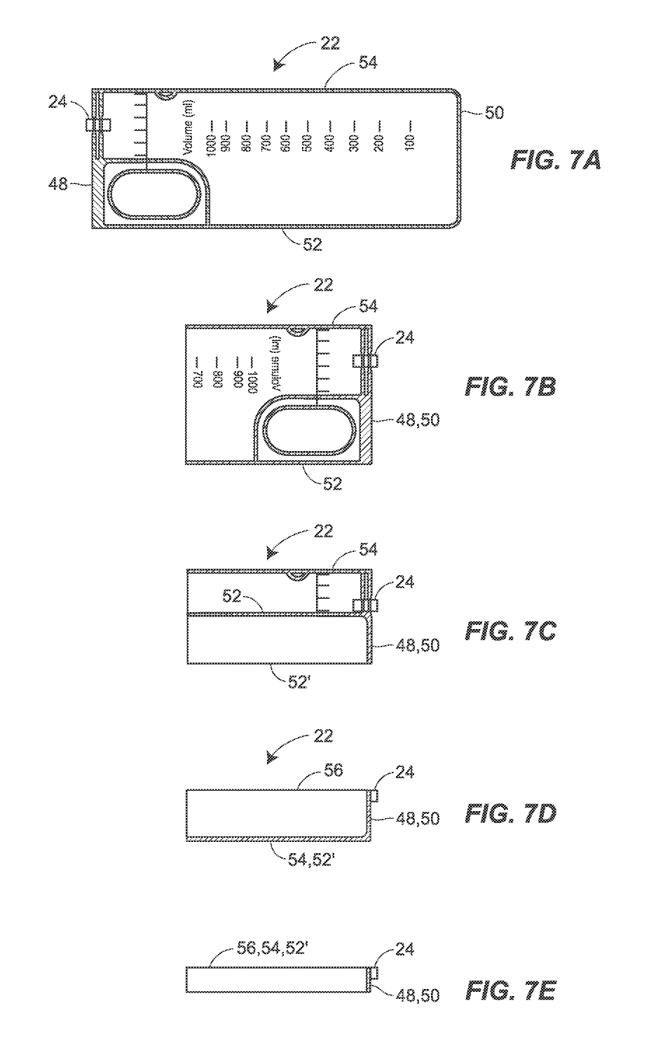

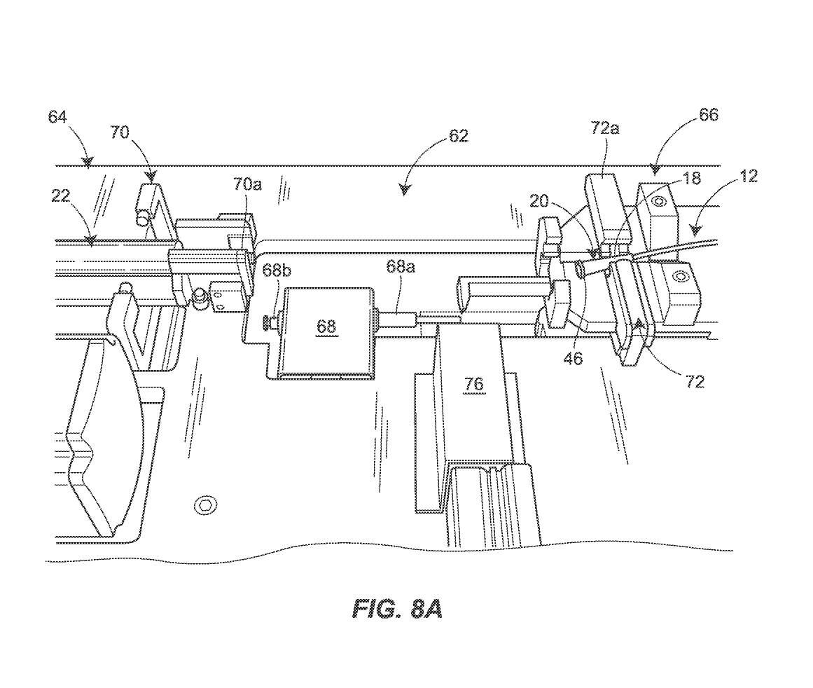

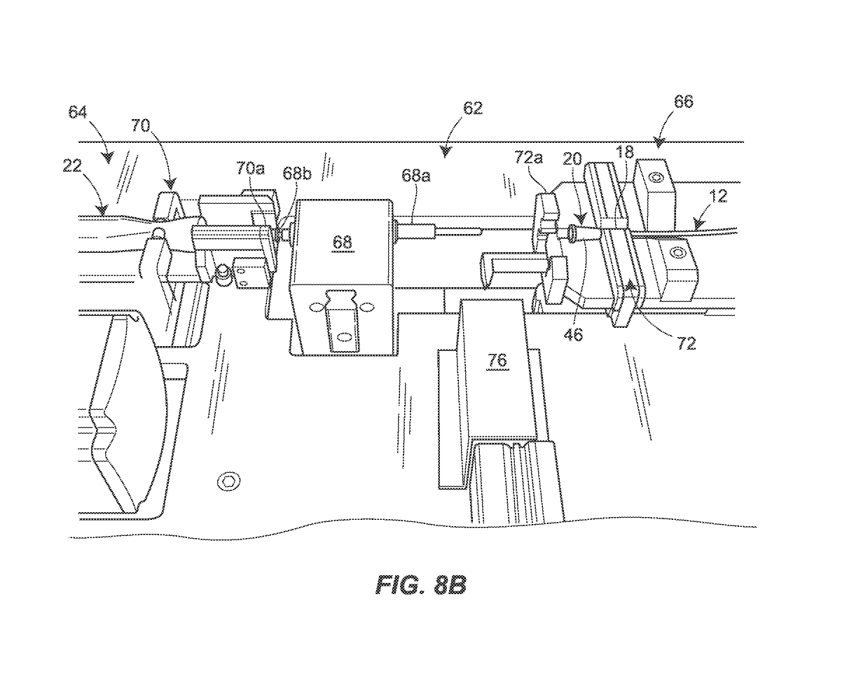

In the illustrations given, and with reference first to FIGS. 1 and 2, the reference numeral 10 designates generally a urine collection assembly in accordance with the present disclosure. The urine collection assembly 10 includes a catheter generally designated 12 having a proximal insertion end 14 and a distal end 16. The distal end 16 has only a cylindrical barrel portion 18 of a tapered funnel such as 20 (FIG. 4) integrally associated therewith. The cylindrical barrel portion 18 of the tapered funnel 20 at the distal end 16 of the catheter 12 is best illustrated in the detailed views of FIGS. 3A and 3B. The urine collection assembly 10 also includes a urine collection bag generally designated 22 having a cylindrical attachment port 24. The cylindrical barrel portion 18 is sealed within the cylindrical attachment port 24 (FIG. 3) to provide a closed collection system. Accordingly, the urine collection assembly 10 permits urine drained from the bladder by the catheter 12 to pass through the cylindrical barrel portion 18 and into the urine collection bag 22.

The urine collection bag 22 can be formed of flexible sheet material with the attachment port 24 sealed into the sheet material. The flexible sheet material can comprise two sheets of EVA/polyethylene film sealed about the peripheral edges thereof. The cylindrical attachment port 24 can be a co-extruded tube formed to have an EVA outer surface and a PVC inner surface. Alternatively, it will be understood that other suitable materials now known or hereafter discovered may be suitable for use.

It is advantageous to utilize these materials for the flexible sheet material and the cylindrical attachment port 24. By utilizing EVA/polyethylene film, the flexible sheet material of the urine collection bag 22 is easy to seal substantially entirely about the peripheral edges thereof, and the cylindrical barrel portion 18 may suitably comprise a portion of a tapered funnel such as 20 which is conventionally formed of PVC. As a result, the cylindrical barrel portion 18 and inner surface of the cylindrical attachment port 24 may be formed of PVC.

Since these like materials are utilized and will be in confronting relation when the cylindrical barrel portion 18 is inserted into cylindrical attachment port 24 during manufacture of the urine collection assembly 10, it is possible to utilize spin welding to seal the cylindrical barrel portion 18 within the cylindrical attachment port 24 to these components together while eliminating the need for the use of adhesives or solvents that can produce harmful vapors.

As will be appreciated, the catheter 12 is disposed externally of the urine collection bag 22 in a ready-to-use position after the cylindrical barrel portion 18 has been sealed within the cylindrical attachment port 24. The catheter 12 may advantageously include a hydrated hydrophilic outer surface to further enhance the desirability of the urine collection assembly 10 by ensuring it is ready-to use without any preliminary preparation. Further, the catheter 12 may include an introducer 26 at or near the proximal insertion end 14 of the catheter 12, and it may also include a sleeve 28 substantially covering the catheter 12 to facilitate no-touch handling. The sleeve 28 may be made from polyurethane or other suitable materials now known or hereafter discovered.

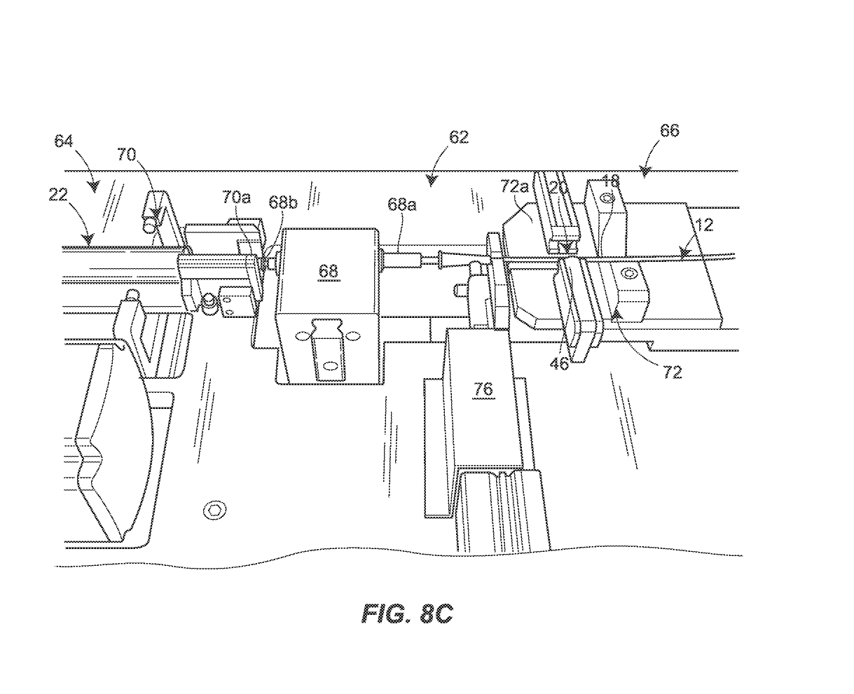

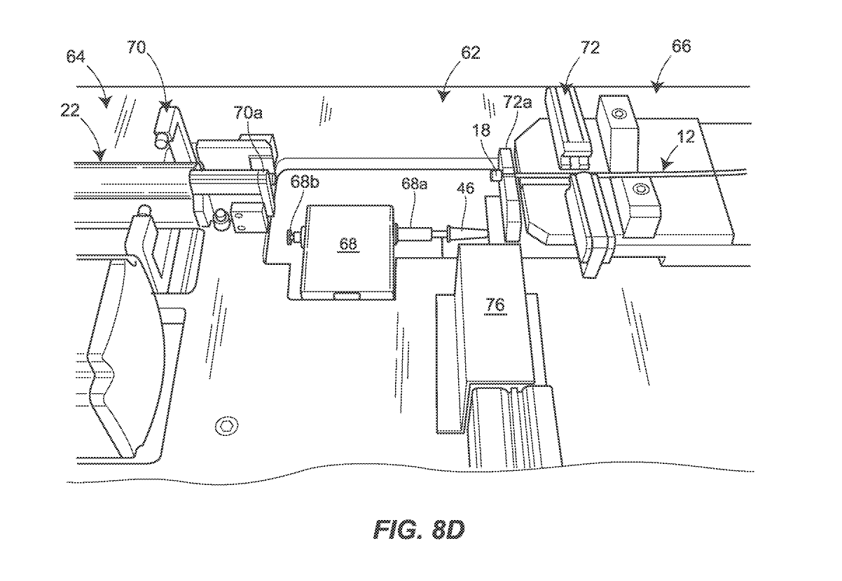

Referring to FIGS. 5 and 5A, the urine collection bag 22 can include an anti-reflux valve 30 sealed to the cylindrical attachment port 24. FIG. 5 illustrates the peripheral seal 32 extending entirely about the periphery of two sheets of flexible material to form the urine collection bag 22. Also, it will be noted that the urine collection bag 22 may include two seal portions 32a and 32b in the region of the cylindrical attachment port 24. With this arrangement, it will be understood that the anti-reflux valve 30 can also be captured by one or both of the seal portions 32a and 32b.

Further, and still referring to FIG. 5, the anti-reflux valve 30 may be formed of two flexible sheets of material advantageously comprising LDPE film. When utilizing flexible sheets or films of this type, it may be advantageous to tack or weld the two sheets or films together at a plurality of discrete locations such as 34. In other words, the tacks or welds may be provided at locations opposite the cylindrical attachment port 24 in substantially equally spaced relation.

With the arrangement illustrated in FIGS. 5 and 5A, the discrete tacks or welds 34 are preferably evenly spaced along the edge 30a of the anti-reflux valve 30 opposite the cylindrical attachment port 24 and extend longitudinally in generally parallel relation to the axis of the cylindrical attachment port 24 to facilitate urine flow into the urine collection bag 22 but prevent reverse urine flow out of the urine collection bag 22, i.e., they form a non-return valve.

FIG. 5 also illustrates two further seals 32c and 32d which define an opening 36 to facilitate handling of the urine collection bag 22. In addition, the urine collection bag 22 may be provided with volume indicia as at 38 indicative of the amount of urine collected therein.

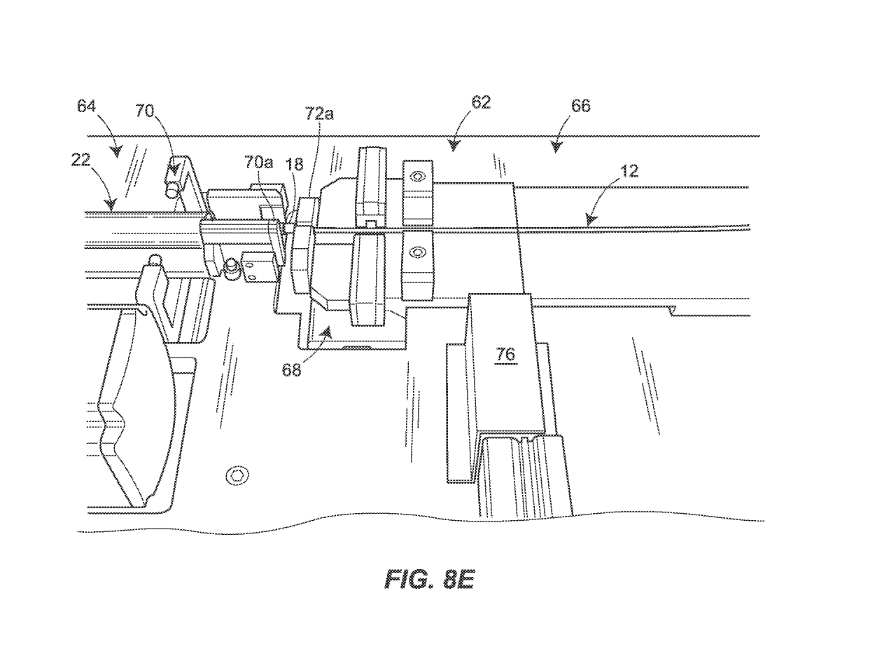

Referring to FIG. 6, it will be appreciated that the urine collection assembly 10 can be provided as a packaged, ready-to-use product. It can then include a gas impermeable package 40, illustrated after opening utilizing a tear strip 42. The catheter 12 and urine collection bag 22 are illustrated after they have been removed from the package 40 and are in a ready-to-use condition.

As illustrated in FIGS. 5 and 6, the urine collection bag 22 is preferably generally rectangular in shape. This permits it to easily be folded at least once generally perpendicular to the direction of the catheter 12 and at least once in the direction of the catheter 12 so that it can be rendered quite compact after folding as illustrated in FIGS. 1 and 2. When the urine collection bag 22 has been folded, a band 44 can be used to hold it in a folded condition.

By folding the urine collection bag 22 in this manner, and utilizing a band 44 to hold it in the folded condition, the urine collection assembly 10 can assume a long, narrow profile suitable for utilizing a long, narrow and compact package such as 40.

In another respect, the present disclosure is directed to a method of manufacturing a packaged ready-to-use urine collection assembly 10. The method comprises the step of providing a catheter 12 having a proximal insertion end 14 and a distal end 16 wherein the catheter 12 can have a hydrophilic outer surface to be hydrated prior to use. The distal end 16 of the catheter 12 is provided with a tapered funnel 20 (FIG. 4) including a cylindrical barrel portion 18 and a conical portion 46 integrally associated therewith. The method also comprises the step of cutting off the conical portion 46 of the tapered funnel 20 and leaving only the cylindrical barrel portion 18 of the tapered funnel 20 on the catheter 12. The method further comprises the step of providing a urine collection bag 22 having a cylindrical attachment port 24 with an opening to receive the cylindrical barrel portion 18 in size for size fashion. It will be understood that due to tolerances there may be some slight size variation between the inside diameter of the cylindrical attachment port 24 and the outside diameter of the cylindrical barrel portion 18. The cylindrical barrel portion 18 of the tapered funnel 20 is placed in the opening defined by the cylindrical attachment port 24 of the urine collection bag 22 in size for size fashion. The method additionally comprises the step of sealing the cylindrical barrel portion 18 of the tapered funnel 20 within the opening defined by the cylindrical attachment port 24 of the urine collection bag 22.

In addition to the foregoing, the method can include the step of providing a gas impermeable package 40 capable of receiving the catheter 12 and the urine collection bag 22 therein, the step of placing the catheter 12, the urine collection bag 22 and a hydrating agent into the gas impermeable package 40, and the step of sealing the gas impermeable package 40 with the catheter 12, the urine collection bag 22 and the hydrating agent therein to complete the ready-to-use assembly.

The method can include the steps of forming the urine collection bag 22 of a flexible sheet material and sealing the attachment port 24 into the sheet material. It can also include the step of using two sheets of EVA/polyethylene film for the sheet material and sealing the two sheets about the peripheral edges 32. It can also include the step of providing a co-extruded tube for the cylindrical attachment port 24 formed with an EVA outer surface and PVC inner surface.

Preferably, the cylindrical barrel portion 18 of the tapered funnel 20 is formed of PVC and the cylindrical barrel portion 18 is sealed within the opening defined by the cylindrical attachment port 24 by spin welding. Still additionally, the spin welding step is advantageously performed by holding the catheter 12 in a fixed or stationary position and then causing the urine collection bag 22 to spin relative to the stationary catheter 12.

The spin welding step can include spinning the urine collection bag 22 relative to the catheter 12 at a rotational speed of between 1900 and 3000 RPMs, depending upon the size of the catheter, and spinning the urine collection bag 22 relative to the catheter 12 for between 14 and 16 revolutions, again depending upon the size of the catheter.

The step of providing a urine collection bag 22 may include providing it in a generally rectangular shape, folding the urine collection bag 22 at least once in the direction of the catheter 12, folding the urine collection bag 22 at least once generally perpendicular to the direction of the catheter 12, performing the spin welding step, again folding the urine collection bag 22 in the direction of the catheter 12, and banding the urine collection bag 22 with a band 44 in a folded condition for placement a package such as 40.

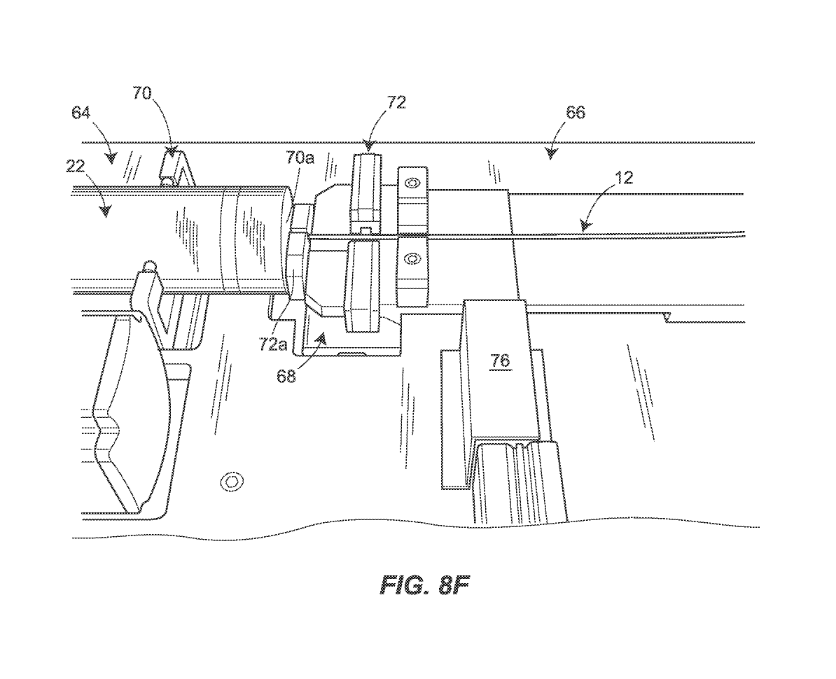

In this connection, FIGS. 7A-7e illustrate a urine collection bag folding process in accordance with the disclosure. The urine collection bag 22 is laid flat in order to initiate the folding process (FIG. 7A), and the urine collection bag 22 is folded in half in the direction of the cylindrical attachment port 24 (and, thus, in the direction of the catheter) so the left edge 48 containing the cylindrical attachment port 24 meets the right edge 50 (FIG. 7B). Next, the bottom edge 52 of the urine collection bag 22 is folded up in a direction generally perpendicular to the cylindrical attachment port 24 (and, thus, generally perpendicular to the direction of the catheter) to touch the bottom of the cylindrical attachment port 24 and define a new bottom edge 52′ after folding (FIG. 7C). Next, the top edge 54 of the urine collection bag 22 is folded down in a direction generally perpendicular to the cylindrical attachment port 24 (and, thus, generally perpendicular to the direction of the catheter) to meet the bottom edge 52′ and define a new top edge 56 after folding (FIG. 7D). Finally, the edges 54, 52′ which meet at the bottom of the urine collection bag 22 in FIG. 7D are folded up in a direction generally perpendicular to the cylindrical attachment port 24 (and, thus, generally perpendicular to the direction of the catheter) to meet the top edge 56 after which the urine collection bag is ready for spin welding.

After the cylindrical barrel portion 18 of a tapered funnel 20 on a catheter 12 has been sealed to the inner surface of the cylindrical attachment port 24 by spin welding (FIG. 3), the urine collection bag can be folded in the direction of the catheter 12 as at 58 and 60 (FIGS. 1 and 2) and banded with a band 44 in the folded condition shown for placement in a package such as 40 (FIG. 6).

Further aspects of the method can include the steps of providing an introducer 26 at or near the proximal insertion end 14 of the catheter 12 and a sleeve 28 substantially covering the catheter 12. The method can also include the step of sealing an anti-reflux valve 30 at a location within the urine collection bag 22 to the cylindrical attachment port 24 and/or sealing the anti-reflux valve 30 to the urine collection bag 22 such as by the seals 32a and 32b. Still further, the method can include the step of providing liquid water as the hydrating agent in the package 40 and including isolating the liquid water from the hydrophilic outer surface of the catheter.

When isolated liquid water is used as the hydrating agent, the method still additionally includes the step of delaying distribution of the gas impermeable package 40 before use of the catheter 12 for a period of time sufficient: i) to form a 100% relative humidity atmosphere within the sealed gas impermeable package 40, and ii) for the hydrophilic outer surface of the catheter 12 to be hydrated so the catheter 12 is ready for use.

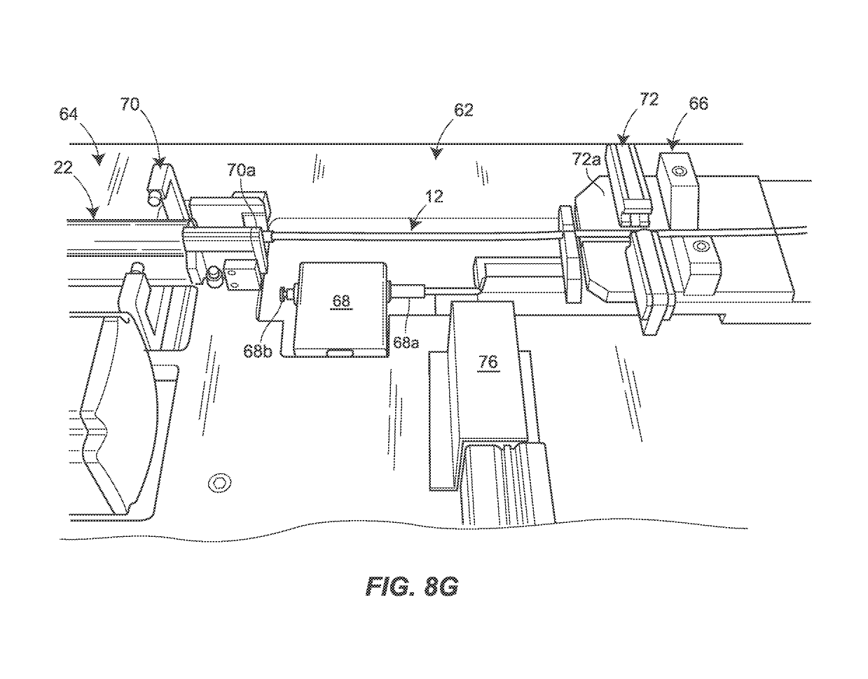

Referring to FIGS. 8A-8G, the reference numeral 62 designates generally an apparatus for performing certain steps of the method of manufacturing a urine collection assembly 10 in accordance with the present disclosure. The apparatus 62 includes a receiving station 64 for the urine collection bag 22 and a receiving station 66 for the conventional catheter 12 wherein the urine collection bag 22 and the catheter 12 are placed in the respective receiving stations 64 and 66 as illustrated in FIG. 8A. After the urine collection bag 22 and the catheter 12 have been placed in the respective receiving stations 64 and 66, a station 68 which supports a mandrel 68a for the funnel 20 on the catheter 12 and which supports a positioning element 68b for the cylindrical attachment port 24 on the urine collection bag 22 is raised as shown in FIG. 8B. It will also be noted that a urine collection bag gripping element 70 is provided for gripping the urine collection bag 22 and a funnel gripping element 72 is provided for gripping the funnel 20 on the catheter 12 (FIG. 8B). By comparing FIGS. 8A and 8B, it can be seen that the gripping elements 70 and 72 undergo linear reciprocating movement between non-gripping positions (FIG. 8A) and positions in which they grip the urine collection bag 22 and the funnel 20 on the catheter 12, respectively (FIG. 8B). In addition, it should be noted that the receiving station 66 and the gripping element 70 are each mounted for movement toward and away from the station 68 for reasons that will become clear in the description which follows.

Referring to FIG. 8C, the apparatus 62 is shown in a position after the gripping element 70 has moved the urine collection bag 22 toward the station 68 to a location where the positioning element 68b properly positions the cylindrical attachment port 24. It is also shown in a position after the receiving station 66 has moved the catheter 12 to properly position the funnel 20 on the mandrel 68a. At this point, the funnel gripping element 72 releases the funnel 20, the station 66 moves away from the station 68, and a second funnel gripping element 72a moves to grip the cylindrical barrel portion 18 of the funnel 20. Further, it will be noted that a cutting element 74 has been moved linearly from within a guard 76 to a position where it can cut off the conical portion 46 leaving only the cylindrical barrel portion 18 of the funnel 18 (see, also, FIG. 4).

After the conical portion 46 of the funnel 20 has been cut off, the cutting element 74 is withdrawn into the guard 76 and the station 68 is dropped down (as shown in FIG. 8D) for removal of the severed conical portion 46 of the funnel 20 from the mandrel 68a. Next, the station 66 is moved toward the station 64 (as shown in FIG. 8E) until the cylindrical barrel portion 18 of the funnel 20 is disposed within the cylindrical attachment port 24 in size to size fashion. Finally, the second funnel gripping element 72a grips the cylindrical barrel portion 18 of the funnel 20 to maintain it in a stationary position and a second urine collection bag gripping element 70a grips and spins both the cylindrical attachment port 24 and the urine collection bag 22 relative to the catheter 12 (FIG. 8F). More specifically, the second urine collection bag gripping element 70a grips and initiates the spinning of the cylindrical attachment port 24 and the urine collection bag 22 just before the cylindrical barrel portion 18 is inserted into the cylindrical attachment port 24 and, then, it accelerates to full speed after the cylindrical barrel portion 18 has been fully inserted.

Referring to FIG. 8G, the apparatus 62 is shown after spinning which completes the manufacture of the urine collection assembly 10. The stations 64 and 66 are again in their starting positions to repeat the process. The cylindrical barrel portion 18 is bonded to the cylindrical attachment port 24 by spin welding without the use of any adhesives or solvents.

While the foregoing sets forth a detailed description of the urine collection assembly and method of the present disclosure, it will be appreciated that the scope of protection is only to be limited by the appended claims.

1. A urine collection assembly, comprising:
a catheter having a proximal insertion end and a distal end having only a cylindrical barrel portion of a tapered funnel integrally associated therewith;
a urine collection bag having a cylindrical attachment port;
the cylindrical barrel portion being sealed in the cylindrical attachment port to thereby permit urine from the catheter to pass into the urine collection bag.

2. The urine collection assembly of claim 1 wherein the urine collection bag is formed of a flexible sheet material and the attachment port is sealed into the urine collection bag.

3. The urine collection assembly of claim 2 wherein the flexible sheet material comprises two sheets of EVA/polyethylene film sealed substantially entirely about the peripheral edges thereof.

4. The urine collection assembly of claim 2 wherein the cylindrical attachment port is a co-extruded tube formed to have an EVA outer surface and a PVC inner surface.

5. The urine collection assembly of claim 4 wherein the cylindrical barrel portion is formed of PVC and is sealed to the inner surface of the co-extruded tube by spin welding.

6. The urine collection assembly of claim 1 wherein the catheter is disposed externally of the urine collection bag and includes a hydrated hydrophilic outer surface.

7. The urine collection assembly of claim 1 including an introducer at or near the proximal insertion end of the catheter and a sleeve substantially covering the catheter.

8. The urine collection assembly of claim 1 including an anti-reflux valve sealed to the cylindrical attachment port within the urine collection bag.

9. The urine collection assembly of claim 8 wherein the anti-reflux valve is formed of two layers of LDPE film also sealed to the urine collection bag.

10. The urine collection assembly of claim 1 further comprising:
a gas impermeable package containing all said urine collection bag wherein said catheter is disposed externally of the urine collection bag and has a hydrated hydrophilic outer surface;
wherein the cylindrical barrel portion of the tapered funnel is sealed internally within the cylindrical attachment port.

11-17. (canceled)

18. The urine collection assembly of claim 10 wherein the urine collection bag is generally rectangular, is folded at least once generally perpendicular to the direction of the catheter, and at least once in the direction of the catheter and is banded in a folded condition.

19. The urine collection assembly of claim 10 including a vapor atmosphere within the gas impermeable package causing the hydrophilic outer surface of the catheter to be hydrated.

20. A method of manufacturing a urine collection assembly, comprising the steps of:
providing a catheter having a proximal insertion end and a distal end having a tapered funnel including a cylindrical barrel portion and a conical portion integrally associated therewith;
cutting off the conical portion of the tapered funnel and leaving only the cylindrical barrel portion of the tapered funnel integrally associated with the distal end of the catheter;
providing a urine collection bag having a cylindrical attachment port defining an opening sized to receive the cylindrical barrel portion of the tapered funnel in size for size fashion;
placing the cylindrical barrel portion of the tapered funnel into the opening defined by the cylindrical attachment port of the urine collection bag in size for size fashion; and
sealing the cylindrical barrel portion of the tapered funnel within the opening defined by the cylindrical attachment port of the urine collection bag.

21. The method of claim 20 including the steps of forming the urine collection bag of a flexible sheet material and sealing the attachment port into the sheet material.

22. The method of claim 21 including the step of using two sheets of EVA/polyethylene film for the flexible sheet material and sealing the two sheets about the peripheral edges thereof.

23. The method of claim 21 including the step of providing a co-extruded tube formed to have an EVA outer surface and a PVC inner surface for the cylindrical attachment port.

24. The method of claim 23 wherein the cylindrical barrel portion is formed of PVC and the step of sealing the cylindrical barrel portion within the opening is performed by spin welding.

25. The method of claim 24 wherein the spin welding step is performed by holding the catheter in a stationary position and spinning the urine collection bag relative to the catheter.

26. The method of claim 25 including the step of providing the urine collection bag in a generally rectangular shape, folding the urine collection bag at least once generally perpendicular to the direction of the catheter, performing the spin welding step, folding the urine collection bag at least once in the direction of the catheter, and banding the urine collection bag in a folded condition.

27. The method of claim 24 wherein the spin welding step includes spinning the urine collection bag relative to the catheter at a rotational speed of between 1900 and 3000 RPMs.

28. The method of claim 27 wherein the spin welding step includes spinning the urine collection bag relative to the catheter for between 14 and 16 revolutions.

29. The method of claim 20 including the step of providing an introducer at or near the proximal insertion end of the catheter and a sleeve substantially covering the catheter.

30. The method of claim 20 including the step of sealing an anti-reflux valve to the cylindrical attachment port within the urine collection bag.

31. The method of claim 20 further comprising the steps of:
providing a catheter having a hydrophilic outer surface to be hydrated prior to using the catheter;
providing a gas impermeable package capable of receiving the catheter and the urine collection bag therein;
placing the catheter, the urine collection bag and a hydrating agent into the gas impermeable package; and
sealing the gas impermeable package with the catheter, the urine collection bag and the hydrating agent therein.

32-41. (canceled)

42. The method of claim 31 wherein the hydrating agent is liquid water and including the step of isolating the liquid water from the hydrophilic outer surface of the catheter.

43. The method of claim 42 including the step of delaying distribution of the gas impermeable package for use of the catheter for a period of time sufficient to form a 100% relative humidity atmosphere within the sealed gas impermeable package and for the hydrophilic outer surface of the catheter to be hydrated so the catheter is ready for use.
